(19) United States
(12) Plant Patent Application Publication
(10) Pub. No.: US 2003/0221233 P1
(43) Pub. Date: Nov. 27, 2003

(54) Schefflera heptaphylla plant named 'Jungle Gem'

(76) Inventor: Robert Cherry, Kulnura (AU)

Correspondence Address: Paul S. Rooy, 2620 S. Peninsula Dr., Daytona Beach, FL, 32118, US

(21) Appl. No.: 10/154,247

(22) Filed: May 23, 2002

(51) Int. Cl. A01H 5/00

(52) U.S. Cl. PLT/377

(57) ABSTRACT

‘Jungle Gem’, a Schefflera heptaphylla cultivar. ‘Jungle Gem’ is a moderately vigorous, erect, evergreen shrub with glossy green alternate, deeply toothed, ovate terminal leaves. Each leaf is palmately compound (digitate) with 5-7 leaflets. Each leaflet is deeply toothed with 5-9 teeth. The petiole is long and is sheathed at the stem. The stem is rounded, green at first, and aging to brown.

### FIELD OF THE INVENTION

This plant patent application claims foreign priority based on Australian Plant Breeder's Rights application No. 1999/113 filed Apr. 28, 1999.

The present invention comprises a new and distinct cultivar of Schefflera heptaphylla which is named ‘Jungle Gem’. Its genus is Schefflera. Its species is heptaphylla. Its market class is that of potted plants. ‘Jungle Gem’ is intended for use in landscaping, and as a decorative potted plant.

### DESCRIPTION OF PRIOR ART

‘Jungle Gem’ was invented in 1994, in Kulnura, NSW, Australia. Its parents were wild Schefflera heptaphylla. ‘Jungle Gem’ was developed by means of open-pollination followed by seedling selection. Seeds from the wild form of Schefflera heptaphylla were collected in 1989. These seeds were grown in trial beds in Kulnura NSW, Australia. Several hundred seedlings were raised.

Those seedlings exhibiting desirable plant habit were retained and unsuitable selections were discarded in 1992. The remaining four plants, all with nice growth habit, were allowed to open pollinate. The resultant seed was collected and sown in 1994. Several hundred seedlings resulted and were grown-on in pots. There was substantial variation in the progeny.

The selection criteria used for ‘Jungle Gem’ was deeply serrated juvenile foliage, vigor, and color of new growth. Propagation trials via cuttings commenced in 1996 and continued for three years. Several thousand plants were produced in the propagation trials, all of which were stable and uniform in all characteristics. The propagules were identical to the original plant in all distinguishing characteristics, thus establishing the stability of ‘Jungle Gem’.

Comparative Trial:

The wild form of Schefflera heptaphylla was selected as the comparator. The comparator is the parent of ‘Jungle Gem’. No other common forms or varieties are known to cultivation.

The comparative trial was conducted at Kulnura from 1995 to 2001. The plants were raised on their own roots from cuttings. The plants were grown in 250 mm pots in commercial potting mix with 30% shade and overhead watering. All plants were subjected to the same chemical treatments for crop protection and nutrition as required. Twelve plants of each variety were arranged in a randomized block. Observations and measurements were taken from twelve plants of each variety.

Compared to wild Schefflera heptaphylla, ‘Jungle Gem’ exhibits deeply toothed, ovate terminal leaflet shape and margin. Its terminal leaflet mean length is 228 mm, mean width is 62 mm, and mean number of lobes is 5 with standard deviation of 1.6. In contrast, wild Schefflera heptaphylla leaflets are entire, occasionally toothed, and of ovate terminal leaflet shape and margin. Wild Schefflera heptaphylla terminal leaflet's mean length is 204 mm, mean width is 69 mm, and mean number of lobes is 1.6 with standard deviation of 1.0.

Uniformity:

Schefflera heptaphylla ‘Jungle Gem” has been propagated asexually through cuttings for four generations. No off-types have been observed throughout the trial period. The variety is considered to be uniform and stable in all characteristics.

### SUMMARY OF THE INVENTION

The major characteristics of ‘Jungle Gem’ include deeply toothed, ovate terminal leaflet shape and margin, vigor, and color of new growth.

### BRIEF DESCRIPTION OF THE DRAWINGS

The invention, together with the other objects, features, aspects and advantages thereof will be more clearly understood from the following in conjunction with the accompanying drawings.

Two sheets of drawings are provided. Sheet one contains FIGS. 1 and 2. Sheet two contains FIG. 3.

FIG. 1 is a color photograph of a front view of ‘Jungle Gem’.

FIG. 2 is a color photograph of a front view of ‘Jungle Gem’.

FIG. 3 is a color photograph of a close-up top view of ‘Jungle Gem’ leaves.

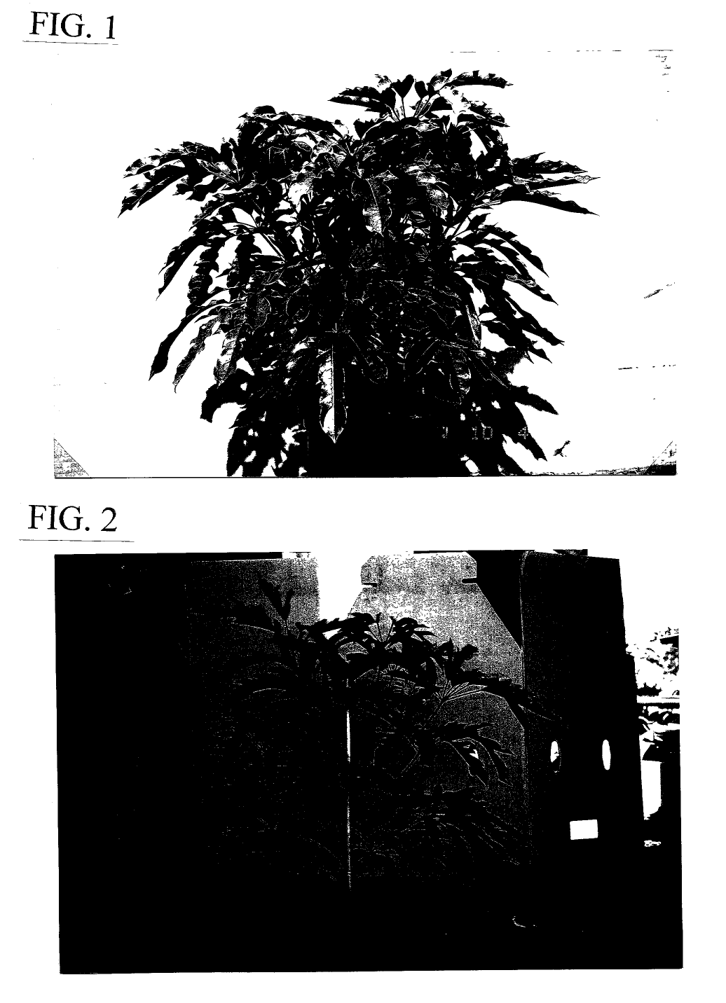

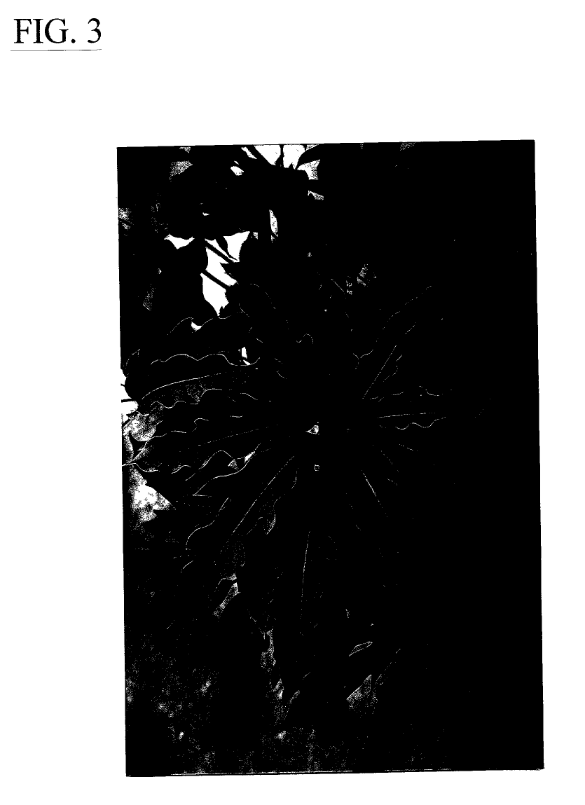

### BOTANICAL DESCRIPTION OF THE PLANT

Schefflera heptaphylla ‘Jungle Gem’ is characterized by its deeply serrated juvenile foliage, vigor, and color of new growth. ‘Jungle Gem’ is a moderately vigorous, growth habit erect, perennial evergreen shrub. Its features are described below.

Stems: Stems are rounded, green at first (RHS 146B), then aging to brown (circa RHS 199B). Lenticels are apparent on immature stems.

Leaves: Leaf arrangement is alternate. Leaf type is palmately compound (digitate), long petioled, with attitude acute.

Petiole: Petiole length is 150-300 mm, with sheathing at stem. Coloration is red-brown circa RHS 177A extending along midrib, becoming paler towards the leaflet apex.

Leaflets: Leaflet number is 6-9 (av. 7). Leaflets are deeply toothed, and there are 5-9 teeth per leaflet (av. 5). The leaflet upper surface is glossy green (RHS 147A) with scattered stellate hairs. The leaflet lower surface is paler green (RHS 147B). Leaflet length (including leaflet stalk and petiolule) is 200-300 mm (av. 228 mm), and leaflet width is 70-90 mm (av. 62 mm). Leaflet shape in cross section is concave. Veins are prominent, the apex is acute-mucronate, and the base is cuneate.

New Growth: Leaves & inflorescence are green-brown (RHS 152C), with puberulent surfaces.

Inflorescence: Inflorescence type is terminal panicle, with large size and strong branching.

Flowers: Flowers are small (7-10 mm in diameter), borne in clusters of 5-12 in small umbels at the end of panicle branches. The corolla exhibits 5-6 petals of pale green (RHS 150C) color. Calyx is absent, the ovary is inferior, and anthers number 5-6.

Buds: Buds are orbicular and yellow-green (RHS 144A) in color. Flowering is infrequent; when it occurs, it does so during spring.

The following is a description of Schefflera heptaphylla ‘Jungle Gem’ during its growth cycle:

Stems: Stems are rounded, green at first (RHS 146B), then aging to brown (circa RHS 199B). Lenticels are apparent on immature stems.

Leaves: New growth leaves are green-brown (RHS 152C), puberulent. alternate, and palmately compound (digitate). Mature leaves are alternate, palmately compound (digitate), long petioled, with attitude acute.

Leaflets: 6-9 (av. 7), deeply toothed, 5-9 teeth per leaflet (av. 5), glossy green above (RHS 147A) with scattered stellate hairs, paler green below (RHS 147B). Leaflet length (incl. leaflet stalk) 200-300 mm (av. 228 mm), width 70-90 mm (av. 62 mm), surface concave, veins prominent, apex acute-mucronate, base cuneate.

All color codes refer to “The Royal Horticultural Society London, 1995Ed.”. Colors and leaf size may vary somewhat depending on horticultural practices such as light levels and fertilizer rates, among other things, without however, any variance in genotype.

I claim:

1. A new and distinct cultivar of Schefflera heptaphylla named ‘Jungle Gem’, substantially as shown and described.
